(19) United States
(12) Patent Application Publication
(10) Pub. No.: US 2012/0009607 A1
(43) Pub. Date: Jan. 12, 2012

(54) DIFFERENTIATING CARDIAC- AND DIABETES MELLITUS-BASED CAUSES OF KIDNEY DAMAGE

(76) Inventors: Georg Hess, Mainz (DE); Andrea Horsch, Mannheim (DE); Dietmar Zdunek, Tutzing (DE)

(21) Appl. No.: 13/242,467

(22) Filed: Sep. 23, 2011

Related U.S. Application Data
(63) Continuation of application No. PCT/EP2010/055861, filed on Apr. 29, 2010.

(30) Foreign Application Priority Data
Apr. 30, 2009 (EP) 09159232.9

(51) Int. Cl. G01N 33/566

(52) U.S. Cl. 435/7.92

(57) ABSTRACT

Disclosed is a method for differentiating in a subject suffering from kidney damage between kidney damage caused by (i) heart failure and/or (ii) diabetes mellitus type 1 or type 2 including the steps of: a) determining the amount of liver-type fatty acid binding protein (L-FABP) and the amount of kidney injury molecule 1 (KIM-1) in a urine-sample of a subject and forming the L-FABP/KIM-1 ratio; b) determining the amount of adiponectin in a urine-sample of said subject; and c) comparing the ratio determined in a) and the amount determined in b) with reference amounts, and establishing the predominant cause of the kidney damage. Also disclosed are a device and a kit for carrying out the method.

### RELATED APPLICATIONS

This application is a continuation of PCT/EP2010/055861 filed Apr. 29, 2010 and claims priority to EP 09159232.9 filed Apr. 30, 2009.

### FIELD

The present invention relates to diagnostic methods and means. Specifically, it relates to a method for differentiating in a subject suffering from kidney damage, preferably chronic kidney damage, more preferably tubular damage and tubular repair, in particular chronic tubular damage and tubular repair, between kidney damage caused by (i) heart failure or (ii) diabetes mellitus.

Moreover, the present invention relates to devices and kits for carrying out said method.

There are two main categories of diabetes mellitus (DM)—type 1 and type 2, which can be distinguished by a combination of features known to the person skilled in the art.

In type 1 diabetes (previously called juvenile-onset or insulin-dependent diabetes), insulin production continuously decreases because of autoimmune pancreatic beta-cell destruction, possibly triggered by environmental exposure in genetically susceptible people. Destruction progresses subclinically over months or years until beta-cell mass decreases to the point that insulin concentrations are no longer adequate to control plasma glucose levels. The type 1 diabetes generally develops in childhood or adolescence and until recently was the most common form diagnosed before age 30; however, it can also develop in adults.

In type 2 diabetes (previously called adult-onset or non-insulin-dependent diabetes), insulin secretion is inadequate. Often insulin levels are very high, especially early in the disease, but peripheral insulin resistance and increased hepatic production of glucose makes insulin levels inadequate to normalize plasma glucose levels. Insulin production then falls, further exacerbating hyperglycemia. The disease generally develops in adults and becomes more common with age. Plasma glucose levels reach higher levels after food intake in older than in younger adults, especially after high carbohydrate loads, and take longer to return to normal, in part because of increased accumulation of visceral and abdominal fat and decreased muscle mass.

Years of poorly controlled diabetes lead to multiple, primarily vascular complications that may affect both small (microvascular) and large (macrovascular) vessels. Microvascular disease underlies the three most common and devastating manifestations of diabetes mellitus: retinopathy, nephropathy, and neuropathy.

Diabetes can lead to kidney damage or renal disorder. A first hint for kidney damage is the presence of protein in urine (micro- or macroalbuminuria) which can be assessed by simple dip stick. The most common test used to date is still creatinine while acknowledging its missing accuracy.

In heart failure (HF), the heart may not provide tissues with adequate blood for metabolic needs, and cardiac-related elevation of pulmonary or systemic venous pressures may result in organ congestion. This condition can result from abnormalities of systolic or diastolic function or, commonly, both.

As cardiac function deteriorates, renal blood flow and GFR decrease, and blood flow within the kidneys is redistributed. The filtration fraction and filtered sodium decrease, but tubular resorption increases, leading to sodium and water retention. Blood flow is further redistributed away from the kidneys during exercise, but renal blood flow improves during rest, possibly contributing to nocturia.

Decreased perfusion of the kidneys activates the renin-angiotensin-aldosterone system, increasing Na and water retention and renal and peripheral vascular tone. These effects are amplified by the intense sympathetic activation accompanying HF.

The renin-angiotensin-aldosterone-vasopressin system produces a cascade of potentially deleterious long-term effects. Angiotensin II worsens HF by causing vasoconstriction, including efferent renal vasoconstriction, and by increasing aldosterone production, which not only enhances Na reabsorption in the distal nephron but also produces myocardial and vascular collagen deposition and fibrosis.

Cardiovascular diseases are increasing with increasing age, so nearly 40% of the population aged 50 already have a detectable cardiovascular disease, which applies for 70% of the population at the age of 75 years (American Heart Association: Heart disease and Stroke statistics—2006, update Dallas AHA 2006; Braunwald Heart disease 8th edition, page 9, FIG. 1-7).

There are manifold causes of the cardiovascular disease which are among others smoking, arterial hypertension, often in connection with metabolic syndrome which is in addition characterized by hyperlipemia, obesity and insulin resistance. Cardiovascular disease may result in heart failure which can be found in 1.5% of all individuals at the age of 50 years and in approximately 10% of individuals at the age of 75 (American Heart Association, Heart Disease and Stroke Statistics 2003, update Dallas AMA 2002).

The worldwide increase of obesity and the metabolic syndrome is believed to feed the diabetes mellitus and cardiovascular endemic. Both disorders are interrelated and are associated with kidney damage requiring further diagnostic differentiation with therapeutic consequences. Current laboratory diagnostic techniques using kidney function tests and the measurement of albumin in urine do not fulfill current diagnostic needs.

Therefore, it is an object of the invention to differentiate in a subject suffering from kidney damage between kidney damage caused by (i) heart failure or (ii) diabetes mellitus.

One of the first hints for kidney damage or renal disorder is the presence of protein in urine (micro- or macroalbuminuria) which can be assessed by simple dip stick. The most common blood test used to date is still creatinine while acknowledging its missing accuracy.

Early identification of subjects suffering from kidney damage and in particular diagnosing its causes is highly desirable.

According to the study of Kollerits et al there is evidence that adiponectin in blood serum may serve as a gender-specific independent predictor of chronic kidney disease progression associated with the metabolic syndrome (Kollerits et al. (2007), Kidney Int. 71(12):1279-86). The role of adiponectin in urine was not studied.

Kamijo et al. (Urinary liver-type fatty acid binding protein as a useful biomarker in chronic kidney disease. Mol. Cell Biochem. 2006; 284) reported that urinary excretion of L-FABP may reflect various kind of stresses that cause tubulointerstitial damage and may be a useful clinical marker of the progression of chronic renal disease.

Van Timmeren et al. (J. Pathol 2007; 212:209-217) reported that tubular kidney injury molecule (KIM-1) is upregulated in renal disease and is associated with renal fibrosis and inflammation. Moreover urinary KIM-1 reflects tissue KIM-1, indicating that it can be used as a non-invasive biomarker in renal disease. One advantage of KIM-1 as a urinary biomarker is the fact that its expression seems to be limited to the injured or diseased kidney (P. Devarajan, Expert Opin. Med, Diagn, (2008) 2(4):387-398).

However, reliable methods for differentiating the causes for kidney damage in a subject suffering from kidney damage have not been described yet.

The technical problem underlying the present invention can be seen as the provision of means and methods for complying with the aforementioned needs. The technical problem is solved by the embodiments characterized in the claims and herein below.

### SUMMARY

Accordingly, the present invention provides a method of differentiating in a subject suffering from kidney damage between kidney damage caused by (i) heart failure and/or (ii) diabetes mellitus type 1 or type 2, based on the comparison of the amounts of liver-type fatty acid binding protein (L-FABP) or a variant thereof, kidney injury molecule 1 (KIM-1) or a variant thereof and adiponectin or a variant thereof, determined in a sample of said subject, preferably determined in a urine sample of the subject, to at least one reference amount.

The method of the present invention may comprise the following steps: a) determining the amounts of liver-type fatty acid binding protein (L-FABP) or a variant thereof, kidney injury molecule 1 (KIM-1) or a variant thereof, adiponectin or a variant thereof, in a sample, preferably a urine-sample of a subject; b) comparing the amounts determined in step a) with reference amounts.

The diagnosis of the predominant cause of the kidney disease may be established based on the information obtained in step b) and preferably based on the information obtained in a) and b).

Step a) may comprise the steps aa) determining the amounts of liver-type fatty acid binding protein (L-FABP) or a variant thereof, kidney injury molecule 1 (KIM-1) or a variant thereof and ab) determining the amounts of adiponectin or a variant thereof.

Accordingly, the present invention provides a method of differentiating in a subject suffering from kidney damage between kidney damage caused by (i) heart failure and/or (ii) diabetes mellitus type 1 or type 2, comprising the steps of:
a) determining the amounts of liver-type fatty acid binding protein (L-FABP) or a variant thereof, kidney injury molecule 1 (KIM-1) or a variant thereof, and adiponectin or a variant thereof, in a urinary sample of a subject;
b) comparing the amounts determined in steps a) to b) with reference amounts;
c) establishing the predominant cause of the kidney damage

It is also provided a method of differentiating in a subject suffering from kidney damage between kidney damage caused by (i) heart failure and/or (ii) diabetes mellitus type 1 or type 2, comprising the steps of:
a) determining the amounts of liver-type fatty acid binding protein (L-FABP) or a variant thereof, kidney injury molecule 1 (KIM-1) or a variant thereof, and adiponectin or a variant thereof, in a urinary sample of a subject;
b) comparing the amounts determined in steps a) to b) with reference amounts;
   whereby the predominant cause of the kidney damage is diagnosed or wherein the comparison of the determined amounts with the reference amounts is indicative for the predominant cause of the kidney damage.

In a further preferred embodiment of the present invention, the L-FABP/KIM-1 ratio is formed. In the context of the present invention, the L-FABP/KIM-1 ratio is also regarded as a “reference amount”.

Moreover, the present invention relates to a device and a kit for carrying out said method.

The method of the present invention is, preferably, an in vitro method. Moreover, it may comprise steps in addition to those explicitly mentioned above. For example, further steps may relate to sample pre-treatments or evaluation of the results obtained by the method.

### DETAILED DESCRIPTION

The term “differentiating” as used herein means to distinguish between a subject which suffers from kidney damage caused by (i) heart failure or kidney damage caused by (ii) diabetes mellitus or kidney damage caused by both diseases (i) and (ii). Said term as used herein accordingly means diagnosing if a subject suffers kidney damage from (i) heart failure or (ii) from diabetes mellitus or from both diseases (i) and (ii).

Diagnosing as used herein refers to assessing or establishing the probability according to which a subject suffers from the diseases referred to in this specification. As will be understood by those skilled in the art, such an assessment is usually not intended to be correct for 100% of the subjects to be diagnosed. The term, however, requires that a statistically significant portion of subjects can be diagnosed to suffer from the said disease (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001.

Diagnosing according to the present invention also includes monitoring, confirmation, subclassification and prediction of the relevant disease, symptoms or risks therefor. Monitoring relates to keeping track of an already diagnosed disease, or complication, e.g., to analyze the progression of the disease or the influence of a particular treatment on the progression of disease or complication. Confirmation relates to the strengthening or substantiating a diagnosis already performed using other indicators or markers. Subclassification relates to further defining a diagnosis according to different subclasses of the diagnosed disease, e.g., defining according to mild and severe forms of the disease. Prediction relates to prognosing a disease or complication before other symptoms or markers have become evident or have become significantly altered.

The term “subject” as used herein relates to animals, preferably mammals, and, more preferably, humans.

However, it is envisaged by the present invention that the subject shall be suffering from kidney damage as specified hereinafter.

The terms “kidney damage”, “kidney disease” or “renal disorders” are well known to the person skilled in the art.

In this context, the term “renal disorder” is considered to relate, preferably, to any dysfunction of the kidney or any dysfunction affecting the capacity of the kidney for waste removal and/or ultrafiltration, in particular any impairment of kidney function as determined by methods known to the person skilled in the art, preferably by GFR and/or creatinine clearance. Examples for renal disorders include congenital disorders and acquired disorders. Examples for congenital renal disorders include congenital hydronephrosis, congenital obstruction of urinary tract, duplicated ureter, horseshoe kidney, polycystic kidney disease, renal dysplasia, unilateral small kidney. Examples for acquired renal disorders include diabetic or analgesic nephropathy, glomerulonephritis, hydronephrosis (the enlargement of one or both of the kidneys caused by obstruction of the flow of urine), interstitial nephritis, kidney stones, kidney tumors (e.g., Wilms tumor and renal cell carcinoma), lupus nephritis, minimal change disease, nephrotic syndrome (the glomerulus has been damaged so that a large amount of protein in the blood enters the urine. Other frequent features of the nephrotic syndrome include swelling, low serum albumin, and high cholesterol), pyelonephritis, renal failure (e.g., acute renal failure and chronic renal failure).

In a preferred embodiment of the present invention, the terms “kidney damage” and “kidney disease” exclude any dysfunction of the kidney or any dysfunction affecting the capacity of the kidney for waste removal and/or ultrafiltration, in particular any impairment of kidney function as determined by methods known to the person skilled in the art, preferably by GFR and/or creatinine clearance. The terms in particular exclude congenital hydronephrosis, congenital obstruction of urinary tract, duplicated ureter, horseshoe kidney, polycystic kidney disease, renal dysplasia, unilateral small kidney, diabetic or analgesic nephropathy, glomerulonephritis, hydronephrosis, interstitial nephritis, kidney stones, kidney tumors (e.g., Wilms tumor and renal cell carcinoma), lupus nephritis, minimal change disease, nephrotic syndrome (swelling, low serum albumin, and high cholesterol), pyelonephritis, renal failure, in particular acute kidney injury (acute renal failure) and chronic kidney disease (chronic renal failure) and cardiorenal syndrome. The terms “kidney damage” and “kidney disease” in particular refer to tubular damage optionally associated with tubular repair. Tubular damage, optionally associated with tubular repair, is also referred to as “progressive tubular disease” in the context of the present invention.

In the context of the present invention kidney damage caused by heart failure is also referred to as “heart failure associated kidney damage”, and kidney damage caused by diabetes mellitus type 1 or type 2 is also referred to as “diabetes mellitus associated kidney damage”.

Renal disorders can be diagnosed by means known to the person skilled in the art. Particularly, renal function (which is used interchangeably with “kidney function” in the context of the present invention) can be assessed by means of the glomerular filtration rate (GFR). For example, the GFR may be calculated by the Cockgroft-Gault or the MDRD formula (Levey 1999, Annals of Internal Medicine, 461-470). GFR is the volume of fluid filtered from the renal glomerular capillaries into the Bowman's capsule per unit time. Clinically, this is often used to determine renal function. The GFR was originally estimated (the GFR can never be determined, all calculations derived from formulas such as the Cockgroft Gault formula of the MDRD formula deliver only estimates and not the “real” GFR) by injecting inulin into the plasma. Since inulin is not reabsorbed by the kidney after glomerular filtration, its rate of excretion is directly proportional to the rate of filtration of water and solutes across the glomerular filter. In clinical practice however, creatinine clearance is used to measure GFR. Creatinine is an endogenous molecule, synthesized in the body, which is freely filtered by the glomerulus (but also secreted by the renal tubules in very small amounts). Creatinine clearance (CrCl) is therefore a close approximation of the GFR. The GFR is typically recorded in millilitres per minute (mL/min). The normal range of GFR for males is 97 to 137 mL/min, the normal range of GFR for females is 88 to 128 mL/min.

GFR is indicative of the kidneys' capacity of water and solutes filtration. A decreased GFR occurs in case of loss of renal tissue (e.g., by necrotic processes). GFR is not indicative for certain renal disorders, e.g., tubular damage. Tubular damage may be present even when GFR is normal.

One of the first hints for renal disorder is the presence of protein in urine (micro- or macroalbuminuria) which can be assessed by simple dip stick. The most common test used to date is still creatinine while acknowledging its missing accuracy.

Chronic kidney disease (CKD) and acute kidney injury (AKI) are known to the person skilled in the art and generally recognized as referring to renal failure as determined by GFR or creatinine clearance.

CKD is known as a loss of renal function which may worsen over a period of months or even years. The symptoms of worsening renal function are unspecific. In CKD glomerular filtration rate is significantly reduced, resulting in a decreased capability of the kidneys to excrete waste products by water and solute filtration. Creatinine levels may be normal in the early stages of CKD. CKD is not reversible. The severity of CKD is classified in five stages, with stage 1 being the mildest and usually causing few symptoms. Stage 5 constitutes a severe illness including poor life expectancy and is also referred to as end-stage renal disease (ESRD), chronic kidney failure (CKF) or chronic renal failure (CRF).

Acute kidney injury (AKI), previously also referred to as acute renal failure (ARF), is a rapid loss of kidney function which may originate from various reasons, including low blood volume and exposure to toxins. Contrary to CKD, AKI can be reversible. AKI is diagnosed on the basis of creatinine levels, urinary indices like blood urea nitrogen (BUN), occurrence of urinary sediment, but also on clinical history. A progressive daily rise in serum creatinine is considered diagnostic of ARF.

The term “cardiorenal syndrome” (also “CRS”) as used in the context of the present invention is to be understood in the sense of the definition established by Ronco et al, in Intensive Care Med. 2008, 34, pages 957-962 and in J. Am. Coll. Cardiol. 2008, 52, p. 1527-1539. Accordingly, CRS refers, in the broadest sense, to a pathophysiologic disorder of the heart and kidneys whereby acute or chronic dysfunction of one of the cited organs may induce acute or chronic dysfunction of the other. The most simple description of CRS is that a relatively normal kidney is dysfunctional because of a diseased heart, assuming that in the presence of a healthy heart, the same kidney would perform normally. 5 subtypes of CRS exist. Type 1 CRS reflects an abrupt worsening of cardiac function (e.g., acute cardiogenic shock or decompensated congestive heart failure) leading to acute kidney injury. Type 2 CRS comprises chronic abnormalities in cardiac function (e.g., chronic congestive heart failure) causing progressive chronic kidney disease. Type 3 CRS consists of an abrupt worsening of renal function (e.g., acute kidney ischemia or glomerulonephritis) causing acute cardiac dysfunction (e.g., heart failure, arrhythmia, ischemia). Type 4 CRS describes a state of chronic kidney disease (e.g., chronic glomerular disease) contributing to decreased cardiac function, cardiac hypertrophy, and/or increased risk of adverse cardiovascular events.

In the context of the present invention, the term “tubular damage” refers to epithelial injury in tubule cells, preferably as a consequence of heart failure and/or diabetes mellitus. The present invention preferably refers to chronic tubular damage. It is believed that in tubular damage tubule cells are ischemic, which may be the consequence of heart failure and/or diabetes mellitus, but it is also believed that tubules have retained their functionality within the kidney entirely or at least to the greatest or a great part. This means that renal function is not impaired or only impaired to a lesser extent, such that CKD or AKI will not or cannot be diagnosed by the methods known in the art, i.e., GFR and/or creatinine clearance. In tubular damage, tubule cells may become dysfunctional, in general by necrosis, and die. However, tubular epithelium regeneration is possible after ischemia and even after necrosis, referred to as “tubular repair” in the context of the present invention. As the present invention preferably refers to chronic tubular injury, it likewise refers to chronic tubular repair or tubular repair from chronic tubular damage.

The term “heart failure” as used herein relates to an impaired systolic and/or diastolic function of the heart. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest. Heart failure, i.e., an impaired systolic and/or diastolic function of the heart, can be determined also by, for example, echocardiography, angiography, szintigraphy, or magnetic resonance imaging. This functional impairment can be accompanied by symptoms of heart failure as outlined above (NYHA class II-IV), although some patients may present without significant symptoms (NYHA I). Moreover, heart failure is also apparent by a reduced left ventricular ejection fraction (LVEF). More preferably, heart failure as used herein is accompanied by a left ventricular ejection fraction (LVEF) of less than 60%, of 40% to 60% or of less than 40%.

The terms “diabetes mellitus type 1” and “diabetes mellitus type 2” have been described in the introductory part of this application and are known to a person skilled in the art.

In type 1 diabetes (previously called juvenile-onset or insulin-dependent diabetes), insulin production continuously decreases because of autoimmune pancreatic beta-cell destruction, possibly triggered by environmental exposure in genetically susceptible people. Destruction progresses subclinically over months or years until beta-cell mass decreases to the point that insulin concentrations are no longer adequate to control plasma glucose levels. The type 1 diabetes generally develops in childhood or adolescence and until recently was the most common form diagnosed before age 30; however, it can also develop in adults.

In type 2 diabetes (previously called adult-onset or non-insulin-dependent diabetes), insulin secretion is inadequate. Often insulin levels are very high, especially early in the disease, but peripheral insulin resistance and increased hepatic production of glucose makes insulin levels inadequate to normalize plasma glucose levels. Insulin production then falls, further exacerbating hyperglycemia. The disease generally develops in adults and becomes more common with age. Plasma glucose levels reach higher levels after food intake in older than in younger adults, especially after high carbohydrate loads, and take longer to return to normal, in part because of increased accumulation of visceral and abdominal fat and decreased muscle mass.

The term “liver-type fatty acid binding protein” (L-FABP, frequently also referred to as FABP1 herein also referred to as liver fatty acid binding protein) relates to a polypeptide being a liver type fatty acid binding protein and to a variant thereof. Liver-type fatty acid binding protein is an intracellular carrier protein of free fatty acids that is expressed in the proximal tubules of the human kidney. For a sequence of human L-FABP, see, e.g., Chan et al.: Human liver fatty acid binding protein cDNA and amino acid sequence, Functional and evolutionary implications, J. Biol. Chem. 260 (5), 2629-2632 (1985) or GenBank Acc. Number M10617.1.

As L-FABP or a variant thereof is preferably determined in a urine sample of the respective subject, is may also be referred to, in the context of the present invention, as “urinary liver-type fatty acid binding protein” or “urinary” L-FABP or U-LFABP.

The term “L-FABP” encompasses also variants of L-FABP, preferably, of human L-FABP. Such variants have at least the same essential biological and immunological properties as L-FABP, i.e., they bind free fatty acids and/or cholesterol and/or retinoids, and/or are involved in intracellular lipid transport. In particular, they share the same essential biological and immunological properties if they are detectable by the same specific assays referred to in this specification, e.g., by ELISA Assays using polyclonal or monoclonal antibodies specifically recognizing the L-FABP. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the human L-FABP, preferably over the entire length of human L-FABP. How to determine the degree of identity is specified elsewhere herein. Variants may be allelic variants or any other species specific homologs, paralogs, or orthologs. Moreover, the variants referred to herein include fragments of L-FABP or the aforementioned types of variants as long as these fragments have the essential immunological and biological properties as referred to above. Such fragments may be, e.g., degradation products of the L-FABP. Further included are variants which differ due to posttranslational modifications such as phosphorylation or myristylation. The term “L-FABP or a variant thereof”, preferably, does not include heart FABP, brain FABP and intestine FABP.

Adiponectin is a polypeptide (one of several known adipocytokines) secreted by the adipocyte. In the art, adiponectin is frequently also referred to as Acrp30 and apM1. Adiponectin has recently been shown to have various activities such as anti-inflammatory, antiatherogenic, preventive for metabolic syndrome, and insulin sensitizing activities. Adiponectin is encoded by a single gene, and has 244 amino acids, the molecular weight is approximately 30 kilodaltons. The mature human adiponectin protein encompasses amino acids 19 to 244 of full-length adiponectin. A globular domain is thought to encompass amino acids 107-244 of full-length adiponectin. The sequence of the adiponectin polypeptide is well known in the art, and, e.g., disclosed in WO/2008/084003.

Adiponectin is the most abundant adipokine secreted by adipocytes. Adipocytes are endocrine secretory cells which release free fatty acids and produce, in addition to adiponectin, several cytokines such as Tumor necrosis factor (TNF) alpha, leptin, and interleukins.

It is generally assumed that adiponectin sensitizes the body to insulin. Decreased adiponectin blood levels are observed in patients with diabetes and metabolic syndrome and are thought to play a key role in insulin resistance (see, e.g., Han et al. Journal of the American College of Cardiology, Vol. 49(5)531-8).

Adiponectin associates itself into larger structures. Three adiponectin polypeptides bind together and form a homotrimer. These trimers bind together to form hexamers or dodecamers. Adiponectin is known to exist in a wide range of multimer complexes in plasma and combines via its collagen domain to create 3 major oligomeric forms: a low-molecular weight (LMW) trimer, a middle-molecular weight (MMW) hexamer, and high-molecular weight (HMW) 12- to 18-mer adiponectin (Kadowaki et al. (2006) Adiponectin and adiponectin receptors in insulin resistance, diabetes, and the metabolic syndrome. J Clin Invest. 116(7): 1784-1792; Rexford S. Ahima, Obesity 2006; 14:2425-2495). Adiponectin has been reported to have several physiological actions, such as protective activities against atherosclerosis, improvement of insulin sensitivity, and prevention of hepatic fibrosis.

Adiponectin as used herein, preferably, relates to total adiponectin, which encompasses low molecular weight adiponectin, mid molecular weight adiponectin and high molecular weight adiponectin. The terms high molecular weight adiponectin, low and mid molecular weight adiponectin and total adiponectin are understood by the skilled person. Preferably, said adiponectin is human adiponectin. Methods for the determination of adiponectin are, e.g., disclosed in US 2007/0042424 A1 as well as in WO/2008/084003. The amount of adiponectin is determined in a urine sample.

The adiponectin referred to in accordance with the present invention further encompasses allelic and other variants of said specific sequence for human adiponectin discussed above. Specifically, envisaged are variant polypeptides which are on the amino acid level preferably, at least 50%, 60%, 70%, 80%, 85%, 90%, 92%, 95%, 97%, 98%, or 99% identical, to human adiponectin, preferably over the entire length of human adiponectin. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs.

Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human adiponectin as long as the said polypeptides have adiponectin properties.

The term “kidney injury molecule-1” (KIM-1) relates to a type 1 membrane protein containing a unique six-cysteine Ig domain and a mucin domain in its extracellular portion. KIM-1 which is the sequence of rat 3-2 cDNA contains an open reading frame of 307 amino acids.

The protein sequence of human cDNA clone 85 also contains one Ig, mucin, transmembrane, and cytoplasmic domain each as rat KIM-1. All six cysteines within the Ig domains of both proteins are conserved. Within the Ig domain, the rat Kim-1 and human cDNA clone 85 exhibit 68.3% similarity in the protein level. The mucin domain is longer, and the cycloplasmic domain is shorter in clone 85 than rat KIM-1, with similarity of 49.3 and 34.8% respectively. Clone 85 is referred to as human KIM-1 (for the structure of KIM-1 proteins see, e.g., Ichimura et al., J Biol Cem, 273 (7), 4135-4142 (1998), in particular FIG. 1). Recombinant human KIM-1 exhibits no cross-reactivity or interference to recombinant rat- or mouse-KIM-1.

KIM-1 mRNA and protein are expressed in high levels in regenerating proximal tubule epithelial cells which cells are known to repair and regenerate the damaged region in the postischemic kidney. KIM-1 is an epithelial cell adhesion molecule (CAM) up-regulated in the cells, which are dedifferentiated and undergoing replication after renal epithelial injury.

A proteolytically processed domain of KIM-1 is easily detected in the urine soon after acute kidney injury (AKI) so that KIM-1 performs as an acute kidney injury urinary biomarker (Expert Opin. Med. Diagn. (2008) 2 (4): 387-398).

KIM-1 referred to in accordance with the present invention further encompasses allelic and other variants of said specific sequence for human KIM-1 discussed above. Specifically, envisaged are variant polypeptides which are on the amino acid level preferably, at least 50%, 60%, 70%, 80%, 85%, 90%, 92%, 95%, 97%, 98%, or 99% identical, to human KIM-1, preferably over the entire length of human KIM-1. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human KIM-1 as long as the said polypeptides have KIM-1 properties. “KIM-1 properties” as used in the context of the present invention refers to inducing dedifferentiation and replication after renal epithelial injury.

Determining the amount of adiponectin or a variant thereof, L-FABP or a variant thereof, KIM-1 or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e., a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers), CBA (an enzymatic Cobalt Binding Assay, available for example on Roche-Hitachi analyzers), and latex agglutination assays (available for example on Roche-Hitachi analyzers).

Preferably, determining the amount of a peptide or polypeptide comprises the steps of (α) contacting a cell capable of eliciting a cellular response the intensity of which is indicative of the amount of the peptide or polypeptide with the said peptide or polypeptide for an adequate period of time, (β) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (α) contacting the peptide with a specific ligand, (optionally) removing non-bound ligand, (β) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include, e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art (e.g., using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available, e.g., from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoroimmuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a peptide or polypeptide may be, also preferably, determined as follows: (α) contacting a solid support comprising a ligand for the peptide or polypeptide as specified above with a sample comprising the peptide or polypeptide and (β) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “sample” refers to a sample of a body fluid, to a sample of separated cells or to a sample from a tissue or an organ. Samples of body fluids can be obtained by well known techniques and include, preferably, samples of blood, plasma, serum, urine, samples of blood, plasma or serum. It is to be understood that the sample depends on the marker to be determined. Therefore, it is encompassed that the polypeptides as referred to herein are determined in different samples. L-FABP or a variant thereof thereof and KIM-1 or a variant thereof and adiponectin or a variant thereof are preferably determined in a urine sample.

The term “forming a ratio” as used herein means calculating in each individual a ratio between the determined amounts of the specified peptides. All ratios were used to calculate a median and respective percentiles to obtain a reference kidney damage information for the target disease.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample or a ratio of amounts is compared to a reference ratio of amounts. The comparison referred to in step (c) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e., automatically provide the desired assessment in a suitable output format.

In general, for determining the respective amounts/amounts or amount ratios allowing to establish the desired diagnosis in accordance with the respective embodiment of the present invention, (“threshold”, “reference amount”), the amount(s)/amount(s) or amount ratios of the respective peptide or peptides are determined in appropriate patient groups. According to the diagnosis to be established, the patient group may, for example, comprise only healthy individuals, or may comprise healthy individuals and individuals suffering from the pathophysiological (state which is to be determined) or may comprise only individuals suffering from the pathophysiological state which is to be determined, or may comprise individuals suffering from the various pathophysiological states to be distinguished, by the respective marker(s) using validated analytical methods. The results which are obtained are collected and analyzed by statistical methods known to the person skilled in the art. The obtained threshold values are then established in accordance with the desired probability of suffering from the disease and linked to the particular threshold value. For example, it may be useful to choose the median value, the 60th, 70th, 80th, 90th, 95th or even the 99th percentile of the healthy and/or non-healthy patient collective, in order to establish the threshold value(s), reference value(s) or amount ratios.

A reference value of a diagnostic marker can be established, and the amount of the marker in a patient sample can simply be compared to the reference value. The sensitivity and specificity of a diagnostic and/or prognostic test depends on more than just the analytical “quality” of the test-they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker of the invention, a distribution of marker amounts for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al, Radiology 143: 29-36 (1982).

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

While exemplary panels are described herein, one or more markers may be replaced, added, or subtracted from these exemplary panels while still providing clinically useful results. Panels may comprise both specific markers of a disease (e.g., markers that are increased or decreased in bacterial infection, but not in other disease states) and/or non-specific markers (e.g., markers that are increased or decreased due to inflammation, regardless of the cause; markers that are increased or decreased due to changes in hemostasis, regardless of the cause, etc.). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

The term “reference amounts” as used herein in this embodiment of the invention refers to amounts of the polypeptides which allow differentiating in a subject suffering from kidney damage between kidney damage caused by (i) heart failure and/or (ii) diabetes mellitus type 1 or type 2,

Therefore, the reference amounts will in general be derived from subjects known to be physiologically healthy, or subjects known to suffer from kidney damage, or subjects suffering from diabetes mellitus 1 or 2, or subjects suffering from diabetes mellitus 1 or 2 and known to suffer from kidney damage, and/or subjects suffering from heart failure, and/or subjects suffering form heart failure and known to suffer from kidney damage.

Accordingly, the term “reference amount” as used herein either refers to an amount which allows diagnosing kidney damage in a subject with diabetes mellitus and/or allows diagnosing kidney damage in a subject with heart failure or suspected to suffer from heart failure. The comparison with reference amounts permits to differentiate between the two types of individuals In the present invention, “reference amount” also refers to the ratio L-FABP/KIM-1 and the ratio L-FABP/adiponectin.

Reference amounts for L-FABP or a variant thereof and KIM-1 or a variant and adiponectin or a variant thereof may be derived from subjects as defined above in the present invention which suffer from diabetes mellitus, and where the subject was diagnosed to suffer from kidney damage, preferably tubular kidney damage and tubular kidney repair, in particular chronic tubular kidney damage and tubular kidney repair, and/or from subjects as defined above in the present invention which suffer from heart failure or are suspected to suffer from heart failure, and where the subject was diagnosed to suffer from kidney damage, preferably tubular kidney damage and tubular kidney repair, in particular chronic tubular kidney damage and tubular kidney repair, and/or from subjects suffering from diabetes mellitus and heart failure and where the subject was diagnosed to suffer from kidney damage. The amounts of the respective peptide serving for establishing the reference amounts can be determined prior to the diagnosis established in accordance with the present invention.

In all embodiments of the present invention, the amount/amounts of the respective markers used therein (L-FABP or a variant thereof and KIM-1 or a variant thereof) are determined by methods known to the person skilled in the art.

In order to test if a chosen reference value yields a sufficiently safe diagnosis of patients suffering from the disease of interest, one may for example determine the efficiency (E) of the methods of the invention for a given reference value using the following formula:

E=(TP/TO)×100;

wherein TP=true positives and TO=total number of tests=TP+FP+FN+TN, wherein FP=false positives; FN=false negatives and TN=true negatives. E has the following range of values: 0<E<100). Preferably, a tested reference value yields a sufficiently safe diagnosis provided the value of E is at least about 50, more preferably at least about 60, more preferably at least about 70, more preferably at least about 80, more preferably at least about 90, more preferably at least about 95, more preferably at least about 98.

The diagnosis if individuals are healthy or suffer from a certain pathophysiological state is made by established methods known to the person skilled in the art. The methods differ in respect to the individual pathophysiological state.

The algorithms to establish the desired diagnosis are laid out in the present application, in the passages referring to the respective embodiment, to which reference is made.

Accordingly, the present invention also comprises a method of determining the threshold amount indicative for a physiological and/or a pathological state and/or a certain pathological state, comprising the steps of determining in appropriate patient groups the amounts of the appropriate marker(s), collecting the data and analyzing the data by statistical methods and establishing the threshold values.

The term “about” as used herein refers to +/−20%, preferably +/−10%, preferably, +/−5% of a given measurement or value.

It is to be understood that if a reference from a subject is used which suffers from a disease or combination of diseases, an amount of a peptide or protein in a sample of a test subject being essentially identical to said reference amount shall be indicative for the respective disease or combination of diseases. The reference amount applicable for an individual subject may vary depending on various physiological parameters such as age, gender, or subpopulation. Moreover, the reference amounts, preferably define thresholds. Thus, a suitable reference amount may be determined by the method of the present invention from a reference sample to be analyzed together, i.e., simultaneously or subsequently, with the test sample. A suitable technique may be to determine the median of the population for the peptide or polypeptide amounts to be determined in the method of the present invention.

KIM-1 and L-FABP are urinary biomarkers which are increased expressed in the proximal tubule epithelial cells in the postischemic kidney.

As L-FABP is considered a biomarker of tubular damage and KIM-1 is believed an indicator of tubular repair, the ratio of both markers reflects evidence of disease progression. As adiponectin appears to be an indicator of “glomerular health”, combined determination of these markers disclose relevant information of pathogenic kidney processes.

Based on the comparison of the L-FABP/KIM-1 ratio formed from the amounts of L-FABP or a variant thereof and KIM-1 or a variant thereof determined in step a) and the amount of adiponectin or a variant thereof determined in step b) and the corresponding reference amounts, subjects suffering from kidney damage caused by (i) heart failure and/or (ii) diabetes mellitus type 1 or type 2 can be identified.

The term “reference amount” as used herein refers to an amount which allows assessing whether kidney damage is caused by (i) heart failure and/or (ii) diabetes mellitus by a comparison as referred to above. Accordingly, the reference may either be derived from a subject suffering from (i) heart failure or (ii) diabetes mellitus type 1 or type 2.

Advantageously, it has been found that a combination of L-FABP or a variant thereof, KIM-1 or a variant thereof and adiponectin or a variant thereof as biomarkers, in particular the ratio of the amounts of L-FABP/KIM-1 in combination with the amount of adiponectin or a variant thereof present in a urine-sample of a subject suffering from a kidney damage, allow for a differential diagnosis with respect to the cause of said symptom in a reliable and efficient manner. Moreover, it has been found that the concentrations of said biomarkers do not correlate. Thus, each of said biomarkers is statistically independent from each other. Thanks to the present invention, subjects can be more readily and reliably diagnosed and subsequently treated according to the result of the said differential diagnosis.

According to the method of the present invention a reference amount of <0.23 μg/g creatinine for adiponectin or a variant thereof, preferable <0.20 μg/g, more preferable <0.15 μg/g, in particular <0.10 μg/g, and a L-FABP/KIM-1 ratio of <18, preferable <16, more preferable <14, in particular <12, are indicative for (i) heart failure.

A reference amount of >0.23 μg/g creatinine for adiponectin or a variant thereof, preferable >0.30 μg/g, more preferable >0.40 μg/g, in particular >0.50 μg/g, and a L-FABP/KIM-1 ratio of >18, preferable >20, more preferable >22, in particular >24, are indicative for (ii) diabetes mellitus type 1 or type 2.

In case the determined amounts of adiponectin or a variant thereof are approximately 0.23 μg/g creatinine and the L-FABP/KIM-1 ratio is approximately 18, the subject shall suffer from heart failure accompanied by diabetes mellitus type 1 or type 2.

Diabetes type 1 and diabetes type 2 represent major risk factors for the development of cardiovascular disorders. It is well known to the person skilled in the art that cardiovascular disease can be symptomatic or asymptomatic and may lead to heart failure. However cardiovascular disease and heart failure may be present in an individual unrelated to diabetes even if diabetes mellitus has been diagnosed in this individual. Also preferably obese individuals may have insulin resistance but no overt diabetes mellitus and suffer from heart failure. Each of these individuals may have detectable kidney damage according to the present invention; however, the underlying cause may be clinically not apparent. Thus the present invention aids in terms of assigning kidney damage to the underlying disease that is the cause of kidney damage and thus guides therapy decision (see below).

The present invention also provides a method of deciding in a subject suffering from kidney damage on a suitable therapy in dependence of its cause (i) heart failure or (ii) diabetes mellitus type 1 or type 2 based on the comparison of the amounts of liver-type fatty acid binding protein (L-FABP) or a variant thereof, kidney injury molecule 1 (KIM-1) or a variant thereof and adiponectin or a variant thereof, determined in a sample of said subject, preferably determined in a urine sample of the subject, to at least one reference amount.

The method of the present invention may comprise the following steps: a) determining the amounts of liver-type fatty acid binding protein (L-FABP) or a variant thereof, kidney injury molecule 1 (KIM-1) or a variant thereof, adiponectin or a variant thereof, in a sample, preferably a urine-sample of a subject; b) comparing the amounts determined in step a) with reference amounts.

The decision on a suitable therapy may be established based on the information obtained in step b) and preferably based on the information obtained in a) and b).

The present invention therefore also provides a method of deciding, in a subject suffering from kidney damage, on a suitable therapy in dependence of its cause (i) heart failure or (ii) diabetes mellitus type 1 or type 2, comprising a) determining the amount of liver-type fatty acid binding protein (L-FABP) or a variant thereof and the amount of kidney injury molecule 1 (KIM-1) or a variant thereof in a urine-sample of a subject;
b) determining the amount of adiponectin or a variant thereof in a urine-sample of said subject; and
c) comparing the ratio determined in a) and the amount determined in b) with reference amounts and establishing the predominant cause of the kidney damage, and
d) deciding on the suitable therapy.

In a preferred embodiment of the present invention, the L-FABP/KIM-1 ratio is formed.

A therapy for a subject suffering from kidney damage caused by diabetes mellitus type 2 is known to the skilled artisan and comprises for example administration of metformin in a suitable dose. Alternatively or in addition, the term relates to life style recommendations given to a subject and/or nutritional diets, preferably, in combination with glucose level control. A therapy for a subject suffering from kidney damage caused by heart failure is also known to the skilled artisan and comprises for example administration of furosemid in a suitable dose. A therapy for both diseases is the administration of blood pressure lowering drugs, most preferably, aspirin, statins, ACE inhibitors and angiotensin II receptor blockers (ABR) (see Eddy 2005, Advances in Chronic Kidney Diseases 12(4):353-365).

The terms “suitable therapy” and “susceptible” as used herein means that a therapy applied to a subject will inhibit or ameliorate the progression of heart failure or diabetes mellitus or its accompanying symptoms. It is to be understood assessment for susceptibility for the therapy will not be correct for all (100%) of the investigated subjects. However, it is envisaged that at least a statistically significant portion can be determined for which the therapy can be successfully applied. Whether a portion is statistically significant can be determined by techniques specified elsewhere herein.

In respect to a suitable therapy for heart failure associated kidney damage and diabetes mellitus associated kidney damage, reference is made to the co-pending applications “Means and methods for diagnosing a diabetes mellitus associated kidney damage in individuals in need of a suitable therapy” claiming priority of 30 Apr. 2009, of the European Patent Application 09 159 233.7, and “Means and methods for diagnosing a heart failure associated kidney damage in individuals in need of a suitable therapy” claiming priority of 30 Apr. 2009, of the European Patent Application 09 159 234.5, of Roche Diagnostics, the disclosures of which in respect to a suitable therapy are incorporated by reference.

Accordingly, the present invention relates to a method for diagnosing myocardial infarction in a subject comprising at least one of the following steps:
a) determining the amounts of a natriuretic peptide and/or troponin T in a sample of the subject;
b) comparing the amounts determined in step a) with reference amounts; and
c) diagnosing myocardial infarction.

Moreover, the present invention also envisages kits and devices adapted to carry out the method of the present invention.

Furthermore, the present invention relates to a device for differentiating in a subject suffering from kidney damage between kidney damage caused by (i) heart failure and/or (ii) diabetes mellitus type 1 or type 2 comprising:
a) means for determining the amount of liver-type fatty acid binding protein (L-FABP) or a variant thereof and the amount of kidney injury molecule 1 (KIM-1) or a variant thereof in a urine-sample of a subject and forming the L-FABP/KIM-1 ratio;
b) means for determining the amount of adiponectin or a variant thereof in a urine-sample of said subject; and
c) means for comparing the ratio determined by the means of a) and the amount determined by the means of b) with reference amounts,
   whereby the device is adapted for establishing the predominant cause of the kidney damage.

The term “device” as used herein relates to a system of means comprising at least the aforementioned means operatively linked to each other as to allow the differentiation. Preferred means for determining the amount of a one of the aforementioned polypeptides as well as means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the polypeptides in an applied sample and a computer unit for processing the resulting data for the evaluation. The computer unit, preferably, comprises a database including the stored reference amounts or values thereof recited elsewhere in this specification as well as a computer-implemented algorithm for carrying out a comparison of the determined amounts for the polypeptides with the stored reference amounts of the database. Computer-implemented as used herein refers to a computer-readable program code tangibly included into the computer unit. Alternatively, where means such as test stripes are used for determining the amount of the peptides or polypeptides, the means for comparison may comprise control stripes or tables allocating the determined amount to a reference amount. The test stripes are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the polypeptides referred to herein, Plasmon surface resonance devices, NMR spectrometers, mass-spectrometers etc.) and/or evaluation units/devices referred to above in accordance with the method of the invention.

Moreover the present invention is concerned with a kit adapted to carry out the method of the present invention, and thus for differentiating in a subject suffering from kidney damage between kidney damage caused by (i) heart failure and/or (ii) diabetes mellitus type 1 or type 2, said kit comprising instructions for carrying out the said method, and
a) means for determining the amount of liver-type fatty acid binding protein (L-FABP) or a variant thereof and the amount of kidney injury molecule 1 (KIM-1) or a variant thereof in a urine-sample of a subject and forming the L-FABP/KIM-1 ratio;
b) means for determining the amount of adiponectin or a variant thereof in a urine-sample of said subject; and
c) means for comparing the ratio determined by the means of a) and the amount determined by the means of b) with reference amounts,
   whereby the kit is adapted for establishing the predominant cause of the kidney damage.

The term “kit” as used herein refers to a collection of the aforementioned compounds, means or reagents of the present invention which may or may not be packaged together. The components of the kit may be comprised by separate vials (i.e., as a kit of separate parts) or provided in a single vial. Moreover, it is to be understood that the kit of the present invention is to be used for practicing the methods referred to herein above. It is, preferably, envisaged that all components are provided in a ready-to-use manner for practicing the methods referred to above. Further, the kit preferably contains instructions for carrying out the said methods. The instructions can be provided by a user's manual in paper- or electronic form. For example, the manual may comprise instructions for interpreting the results obtained when carrying out the aforementioned methods using the kit of the present invention.

How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test stripes are used for determining the amount of the peptides or polypeptides, the means for comparison may comprise control stripes or tables allocating the determined amount to a reference amount. The test stripes are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the KIM-1 or a variant thereof, L-FABP or a variant thereof and a cardiac troponin. Plasmon surface resonance devices, NMR spectrometers, mass-spectrometers etc.) or evaluation units/devices referred to above in accordance with the method of the invention.

The present invention also relates to the use of a kit or device for determining the amount of KIM-1 or a variant thereof, L-FABP or a variant thereof and adiponectin or a variant thereof in a sample of a subject, comprising means for determining the amount of KIM-1 or a variant thereof, L-FABP or a variant thereof and adiponectin or a variant thereof and/or means for comparing the amount of KIM-1 or a variant thereof, L-FABP or a variant thereof and adiponectin or a variant thereof to at least one reference amount for: differentiating in a subject suffering from kidney damage between kidney damage caused by (i) heart failure and/or (ii) diabetes mellitus type 1 or type 2; and/or deciding in a subject suffering from kidney damage, on a suitable therapy in dependence of its cause (i) heart failure or (ii) diabetes mellitus type 1 or type 2.

The present invention also relates to the use of: an antibody against KIM-1 or a variant thereof, an antibody against L-FABP or a variant thereof and an antibody against adiponectin or a variant thereof, and/or of means for determining the amount of KIM-1 or a variant thereof, L-FABP or a variant thereof and adiponectin or a variant thereof, and/or of means for comparing the amount of KIM-1 or a variant thereof, L-FABP or a variant thereof and adiponectin or a variant thereof to at least one reference amount, for the manufacture of a diagnostic composition for: differentiating in a subject suffering from kidney damage between kidney damage caused by (i) heart failure and/or (ii) diabetes mellitus type 1 or type 2; and/or deciding in a subject suffering from kidney damage, on a suitable therapy in dependence of its cause (i) heart failure or (ii) diabetes mellitus type 1 or type 2.

The present invention also relates to the use of: an antibody against KIM-1 or a variant thereof, an antibody against L-FABP or a variant thereof and an antibody against adiponectin or a variant thereof, and/or of means for determining the amount of KIM-1 or a variant thereof, L-FABP or a variant thereof and adiponectin or a variant thereof, and/or of means for comparing the amount of KIM-1 or a variant thereof, L-FABP or a variant thereof and adiponectin or a variant thereof to at least one reference amount for: differentiating in a subject suffering from kidney damage between kidney damage caused by (i) heart failure and/or (ii) diabetes mellitus type 1 or type 2; and/or deciding in a subject suffering from kidney damage, on a suitable therapy in dependence of its cause (i) heart failure or (ii) diabetes mellitus type 1 or type 2.

All references cited in this specification are herewith incorporated by reference with respect to their entire disclosure content and the disclosure content specifically mentioned in this specification.

The following examples shall merely illustrate the invention. They shall not be construed, whatsoever, to limit the scope of the invention.

### EXAMPLES

### General

In all examples, urinary levels of said biomarkers were determined using the following commercially available immunoassay kits:

L-FABP was determined by using the L-FABP ELISA-Kit from CMIC Co., Ltd, Japan The test is based on an ELISA 2-step assay. L-FABP standard or urine samples are firstly treated with pretreatment solution, and transferred into a L-FABP antibody coated microplate containing assay buffer and incubated. During this incubation, L-FABP in the reaction solution binds to the immobilized antibody. After washing, the 2nd antibody-peroxidase conjugate is added as the secondary antibody and incubated, thereby forming sandwich of the L-FABP antigen between the immobilized antibody and conjugate antibody. After incubation, the plate is washed and substrate for enzyme reaction is added, color develops according to the L-FABP antigen quantity. The L-FABP concentration is determined based on the optical density.

Adiponectin (multimeric) was determined by using the test EIA from ALPCO diagnostics (USA), operating on the principle of a “sandwich” format ELISA. The specific antibodies used in the kit are anti-human adiponectin monoclonal antibodies (MoAbs) directed to two independent epitopes. The specimens are pre-treated as described below, and total adiponectin and individual multimers of adiponectin are determined selectively, directly or indirectly. Multimers of adiponectin are classified into four fractions with this kit:

1) Total adiponectin fraction: “Total-Ad”-assayed directly on the plate
2) High-molecular adiponectin fraction (equivalent of dodecamer-octodecamer): “HMW-Ad”-assayed directly on the plate
3) Middle-molecular adiponectin fraction (equivalent of hexamer): “MMW-Ad”-inferred value obtained by subtracting the concentration of HMW-Ad from the combined concentration of MMW-Ad+HMW-Ad
4) Low-molecular adiponectin fraction (equivalent of trimer including albumin-binding adiponectin): “LMWAd”-inferred value obtained by subtracting the combined concentration of MMW-Ad+HMW-Ad from the total concentration of Ad. The microtiter plate wells have been coated with an anti-human adiponectin monoclonal antibody. Adiponectin in the standards and pretreated specimens are captured by the antibody during the first incubation. Afterwards, a wash step removes all unbound material. Subsequently, an anti-human adiponectin antibody which has been biotin-labeled is added and binds to the immobilized adiponectin in the wells. Subsequently, an anti-human adiponectin antibody which has been biotin-labeled is added and binds to the immobilized adiponectin in the wells. After the second incubation and subsequent wash step, HRP-labeled streptavidin is added. After the third incubation and subsequent wash step, substrate solution is added. Finally, stop reagent is added after allowing the color to develop. The intensity of the color development is read by a microplate reader. The absorbance value reported by the plate reader is proportional to the concentration of adiponectin in the sample.

Human KIM-1 was determined by the Human KIM-1 (catalogue number DY 1750) ELISA Development kit from R&D-Systems, containing a capture antibody (goat anti-human TIM-1) and a detection antibody (biotinylated goat anti-human TIM-1). A seven point standard curve using 2-fold serial dilutions in Reagent Diluent, and a high standard of 2000 pg/mL is recommended.

### Example 1

Patients suffering from diabetes type 1 (a total of 203 patients), diabetes type 2 (a total of 134 patients) and heart failure (a total of 44 patients) were investigated for urine levels of adiponectin, KIM-1 and L-FABP. The afore-mentioned patients suffering from diabetes type 1 or type 2 showed no hints for heart failure as well as the patients suffering from heart failure showed any hints of evident diabetes mellitus type 1 or type 2. All patients were clinically stable, their kidney function was in the normal range as assessed by creatinine levels. Moreover lower urinary tract infection was not detectable in any patient as assessed by the absence of clinical symptoms such as dysuria or pollacisuria and by routine dip stick.

The biomarker median levels of said study and the L-FABP/KIM-1 ratio are summarized in the following table.

TABLE 1
Biomarkers Median Levels

| | Adiponectin | u-L-FABP | KIM-1 | L-FABP/ |
|---|---|---|---|---|
| | μg/g | μg/g | μg/g | KIM-1 |
| | Creatinin | Creatinin | Creatinin | Ratio |
| Heart failure | 0.137 | 7.664 | 0.574 | 13.80 |
| Diabetes | 0.42 | 5.513 | 0.314 | 22.37 |
| mellitus type 1 | | | | |
| Diabetes | 0.33 | 8.21 | 0.38 | 22.55 |
| mellitus type 2 | | | | |

It has been found that in a urine-sample of subjects suffering from heart failure significantly lower amounts of adiponectin are determined with respect to urine samples of subjects suffering from diabetes mellitus type 1 or type 2. Moreover, the L-FABP/KIM-1 ratio determined for subjects suffering from heart failure is significantly lower than the L-FABP/KIM-1 ratio determined for subjects suffering from diabetes mellitus type 1 or type 2.

What is claimed is:

1. A method for differentiating between kidney damage caused by heart failure and kidney damage caused by diabetes mellitus type 1 or type 2 in a subject suffering from kidney damage, the method comprising the steps of:
determining an amount of adiponectin in a urine sample from the subject,
determining an amount of liver-type fatty acid binding protein (L-FABP) and an amount of kidney injury molecule 1 (KIM-1) in a urine sample from the subject and calculating an L-FABP/KIM-1 ratio from the amounts determined, and
comparing the amount of adiponectin determined and the L-FABP/KIM-1 ratio calculated with a reference amount for adiponectin and a reference L-FABP/KIM-1 ratio, wherein a first reference amount for adiponectin and a first reference L-FABP/KIM-1 ratio are derived from patients suffering from kidney damage and heart failure, and a second reference amount for adiponectin and a second reference L-FABP/KIM-1 ratio are derived from patients suffering from kidney damage and diabetes type 1 or 2, wherein a determined amount of adiponectin and a calculated L-FABP/KIM-1 ratio less than the first reference amount and first reference ratio is indicative of heart failure as a cause of the kidney damage, while a determined amount of adiponectin and a calculated L-FABP/KIM-1 ratio greater than the second reference amount and second reference ratio are indicative of diabetes type 1 or 2 as a cause of the kidney damage.

2. The method of claim 1, wherein the first reference amount for adiponectin is 0.20 μg/g creatinine and the first reference L-FABP/KIM-1 ratio is 16.

3. The method of claim 1, wherein the second reference amount for adiponectin is 0.30 μg/g creatinine and the second reference L-FABP/KIM-1 ratio is 20.

4. A method of deciding, for a subject suffering from kidney damage, on a suitable therapy based on whether the kidney damage is caused by heart failure or diabetes mellitus type 1 or type 2, the method comprising:
determining an amount of adiponectin in a urine-sample from the subject,
determining an amount of liver-type fatty acid binding protein (L-FABP) and an amount of kidney injury molecule 1 (KIM-1) in a urine sample from the subject and calculating an L-FABP/KIM-1 ratio,
comparing the L-FABP/KIM-1 ratio and the amount of adiponectin determined with reference amounts for L-FABP/KIM-1 ratio and adiponectin, wherein a first reference amount for adiponectin and a first reference L-FABP/KIM-1 ratio are derived from patients suffering from kidney damage and heart failure and a second reference amount for adiponectin and a second reference L-FABP/KIM-1 ratio are derived from patients suffering from kidney damage and diabetes type 1 or 2,
deciding on a therapy for heart failure if the determined amount of adiponectin and the calculated L-FABP/KIM-1 ratio are less than the first reference amount and first reference ratio, and deciding on a therapy for diabetes if the determined amount of adiponectin and the calculated L-FABP/KIM-1 ratio are greater than the second reference amount and second reference ratio.

5. A device for differentiating between kidney damage caused by heart failure and diabetes mellitus type 1 or type 2 in a subject suffering from kidney damage, the device comprising:
means for determining an amount of liver-type fatty acid binding protein (L-FABP) and an amount of kidney injury molecule 1 (KIM-1) in a urine-sample from the subject,
means for calculating an L-FABP/KIM-1 ratio,
means for determining an amount of adiponectin in a urine-sample from the subject, and
means for comparing the L-FABP/KIM-1 ratio and the amount of adiponectin determined with reference amounts of adiponectin and reference L-FABP/KIM-1 ratios,
whereby the device is adapted for establishing a predominant cause of the kidney damage.

6. A kit for differentiating between kidney damage caused by heart failure and diabetes mellitus type 1 or type 2 in a subject suffering from kidney damage, the kit comprising:
reagents for determining an amount of liver-type fatty acid binding protein (L-FABP) and an amount of kidney injury molecule 1 (KIM-1) in a urine-sample from the subject,
reagents for determining an amount of adiponectin in a urine-sample from the subject, and
instructions for use, including calculation of an L-FABP/KIM-1 ratio and comparison of the amount of adiponectin determined and the L-FABP/KIM-1 ratio calculated to reference amounts for adiponectin and L-FABP/KIM-1.

7. The method of claim 1, wherein the first reference amount for adiponectin is 0.23 μg/g creatinine and the first reference L-FABP/KIM-1 ratio is 18.

8. The method of claim 1, wherein the second reference amount for adiponectin is 0.23 μg/g creatinine and the second reference L-FABP/KIM-1 ratio is 18.

9. The method of claim 1, wherein the first reference amount for adiponectin is 0.15 μg/g creatinine and the first reference L-FABP/KIM-1 ratio is 14.

10. The method of claim 1, wherein the second reference amount for adiponectin is 0.40 μg/g creatinine and the second reference L-FABP/KIM-1 ratio is 22.

11. The method of claim 1, wherein the first reference amount for adiponectin is 0.10 μg/g creatinine and the first reference L-FABP/KIM-1 ratio is 12.

12. The method of claim 1, wherein the second reference amount for adiponectin is 0.50 μg/g creatinine and the second reference L-FABP/KIM-1 ratio is 24.
